SEQUENCE LISTING (24 sequences)

SEQ ID NO: 1 - DNA, 100 bp, Artificial Sequence
  wild-type DNA and DNA with G>A intron 4 mutation
cctctgatggttttgtcctcatccctgacctcaagtggaaccaacagcaggtaaaggtttctggctggaatgtcctgatctctggccaccctgctgtaag

SEQ ID NO: 2 - DNA, 100 bp, Artificial Sequence
  wild-type DNA and DNA with G>A intron 4 mutation
cctctgatggttttgtcctcatccctgacctcaagtggaaccaacagcagataaaggtttctggctggaatgtcctgatctctggccaccctgctgtaag

SEQ ID NO: 3 - DNA, 1508 bp, Artificial Sequence
  mRNA in wild-type and G>4 intron 4 mutation
gggcgtcttcggctcccaccccggggcgtgggcctgggggtccctcttcactgcgcagccacctctagcccgccgctttcgggactctcagccttcttttcccttccctgagggccggccggcaccacaacggggccaaaggcagtaactcggttgaaaggcgcgccctgggacaggtcagcgggctccgccatgcgcggcacgcatcccgcaggagcgggtgccagagcagggtaggccgggagttagcagcccagccaggccgctggactcgggctaggggcggggccagcgcagggggcgcaggcgcacaccgcctccgcggcagcatggcggacgcagctcctcaactaggcaagaggaagcgcgaattggacgtggaggaggcccacgccgccagcacagaggaaaaggaggcaggagttggaaatggtacctgtgctcctgtccgcttaccgttctccggcttcagactgcagaaggtgctgagggagtctgcgcgggacaaaatcattttcctacacgggaaggtgaatgaggcctctggggatggggatggagaggatgccgttgtgatcctggagaagacgccatttcaggtggaacaggtggctcagctcctgacgggcagccctgagctccaattgcagttctccaatgatatctacagcacctatcacttgttccctccaagacaactgaatgatgtaaagacgaccgtggtttaccctgccacagagaaacacctgcagaagtacctgcgccaggacctccgcctgatccgagagacgggagatgactacaggaatattactttaccccacctggagtcccagagcctcagcatccagtgggtgtataacattctcgacaagaaggctgaagcggaccggattgttttcgagaacccagatccctctgatggttttgtcctcatccctgacctcaagtggaaccaacagcagctcgatgacttgtacttgatcgccatctgccatcgccggggcatcagatccctacgcgaccttactccggagcacttgccgctgctcaggaacatcctccaccaggggcaggaggccatcctgcagcgctaccggatgaagggagaccatctgcgagtatacctgcactacctgccctcctactaccacctgcatgtgcacttcaccgccctgggcttcgaggcccccggctcaggcgtggagcgggcccacctgctggctgaggtgatcgagaacttggagtgtgaccctaggcactaccagcagcgcacgctcaccttcgccctcagggctgacgaccccctgctcaagctcttgcaggaggctcagcaaagctgaattaactcaggcagaagagcacagatgtgtgggattgggggaggagtggggacaagattttttatctccaagtgaattttctaaaaatgtattttataccggcttattcctagtattgaataaactagcgggctcaaaaaaaaaaaaaaaaaaaaaaaaaaaaaa

SEQ ID NO: 4 - DNA, 1553 bp, Artificial Sequence
  mRNA in wild-type and G>4 intron 4 mutation
gggcgtcttcggctcccaccccggggcgtgggcctgggggtccctcttcactgcgcagccacctctagcccgccgctttcgggactctcagccttcttttcccttccctgagggccggccggcaccacaacggggccaaaggcagtaactcggttgaaaggcgcgccctgggacaggtcagcgggctccgccatgcgcggcacgcatcccgcaggagcgggtgccagagcagggtaggccgggagttagcagcccagccaggccgctggactcgggctaggggcggggccagcgcagggggcgcaggcgcacaccgcctccgcggcagcatggcggacgcagctcctcaactaggcaagaggaagcgcgaattggacgtggaggaggcccacgccgccagcacagaggaaaaggaggcaggagttggaaatggtacctgtgctcctgtccgcttaccgttctccggcttcagactgcagaaggtgctgagggagtctgcgcgggacaaaatcattttcctacacgggaaggtgaatgaggcctctggggatggggatggagaggatgccgttgtgatcctggagaagacgccatttcaggtggaacaggtggctcagctcctgacgggcagccctgagctccaattgcagttctccaatgatatctacagcacctatcacttgttccctccaagacaactgaatgatgtaaagacgaccgtggtttaccctgccacagagaaacacctgcagaagtacctgcgccaggacctccgcctgatccgagagacgggagatgactacaggaatattactttaccccacctggagtcccagagcctcagcatccagtgggtgtataacattctcgacaagaaggctgaagcggaccggattgttttcgagaacccagatccctctgatggttttgtcctcatccctgacctcaagtggaaccaacagcagataaaggtttctggctggaatgtcctgatctctggccaccctgctctcgatgacttgtacttgatcgccatctgccatcgccggggcatcagatccctacgcgaccttactccggagcacttgccgctgctcaggaacatcctccaccaggggcaggaggccatcctgcagcgctaccggatgaagggagaccatctgcgagtatacctgcactacctgccctcctactaccacctgcatgtgcacttcaccgccctgggcttcgaggcccccggctcaggcgtggagcgggcccacctgctggctgaggtgatcgagaacttggagtgtgaccctaggcactaccagcagcgcacgctcaccttcgccctcagggctgacgaccccctgctcaagctcttgcaggaggctcagcaaagctgaattaactcaggcagaagagcacagatgtgtgggattgggggaggagtggggacaagattttttatctccaagtgaattttctaaaaatgtattttataccggcttattcctagtattgaataaactagcgggctcaaaaaaaaaaaaaaaaaaaaaaaaaaaaaa

SEQ ID NO: 5 - protein, 337 aa, Artificial Sequence
  Protein in wild-type and G>4 intron 4 mutation
MADAAPQLGKRKRELDVEEAHAASTEEKEAGVGNGTCAPVRLPFSGFRLQKVLRESARDKIIFLHGKVNEASGDGDGEDAVVILEKTPFQVEQVAQLLTGSPELQLQFSNDIYSTYHLFPPRQLNDVKTTVVYPATEKHLQKYLRQDLRLIRETGDDYRNITLPHLESQSLSIQWVYNILDKKAEADRIVFENPDPSDGFVLIPDLKWNQQQLDDLYLIAICHRRGIRSLRDLTPEHLPLLRNILHQGQEAILQRYRMKGDHLRVYLHYLPSYYHLHVHFTALGFEAPGSGVERAHLLAEVIENLECDPRHYQQRTLTFALRADDPLLKLLQEAQQS

SEQ ID NO: 6 - protein, 352 aa, Artificial Sequence
  Protein in wild-type and G>4 intron 4 mutation
MADAAPQLGKRKRELDVEEAHAASTEEKEAGVGNGTCAPVRLPFSGFRLQKVLRESARDKIIFLHGKVNEASGDGDGEDAVVILEKTPFQVEQVAQLLTGSPELQLQFSNDIYSTYHLFPPRQLNDVKTTVVYPATEKHLQKYLRQDLRLIRETGDDYRNITLPHLESQSLSIQWVYNILDKKAEADRIVFENPDPSDGFVLIPDLKWNQQQIKVSGWNVLISGHPALDDLYLIAICHRRGIRSLRDLTPEHLPLLRNILHQGQEAILQRYRMKGDHLRVYLHYLPSYYHLHVHFTALGFEAPGSGVERAHLLAEVIENLECDPRHYQQRTLTFALRADDPLLKLLQEAQQS

SEQ ID NO: 7 - DNA, 290 bp, Artificial Sequence
  Wild-type DNA and DNA with C>T Exon 6 Thr316Met missense mutation
ctaggtggggacatggcgccgggcctcaggcagcacggtgactcctgacctgcctgcccctgtctcatcaggaggccatcctgcagcgctaccggatgaagggagaccatctgcgagtatacctgcactacctgccctcctactaccacctgcatgtgcacttcaccgccctgggcttcgaggcccccggctcaggcgtggagcgggcccacctgctggctgaggtgatcgagaacttggagtgtgaccctaggcactaccagcagcgcacgctcaccttcgccctcagg

SEQ ID NO: 8 - DNA, 290 bp, Artificial Sequence
  Wild-type DNA and DNA with C>T Exon 6 Thr316Met missense mutation
ctaggtggggacatggcgccgggcctcaggcagcacggtgactcctgacctgcctgcccctgtctcatcaggaggccatcctgcagcgctaccggatgaagggagaccatctgcgagtatacctgcactacctgccctcctactaccacctgcatgtgcacttcaccgccctgggcttcgaggcccccggctcaggcgtggagcgggcccacctgctggctgaggtgatcgagaacttggagtgtgaccctaggcactaccagcagcgcatgctcaccttcgccctcagg

SEQ ID NO: 9 - protein, 337 aa, Artificial Sequence; identical to SEQ ID NO: 5
  Protein in wild-type and C>T Exon 6 Thr316Met missense mutation

SEQ ID NO: 10 - protein, 337 aa, Artificial Sequence
  Protein in wild-type and C>T Exon 6 Thr316Met missense mutation
MADAAPQLGKRKRELDVEEAHAASTEEKEAGVGNGTCAPVRLPFSGFRLQKVLRESARDKIIFLHGKVNEASGDGDGEDAVVILEKTPFQVEQVAQLLTGSPELQLQFSNDIYSTYHLFPPRQLNDVKTTVVYPATEKHLQKYLRQDLRLIRETGDDYRNITLPHLESQSLSIQWVYNILDKKAEADRIVFENPDPSDGFVLIPDLKWNQQQLDDLYLIAICHRRGIRSLRDLTPEHLPLLRNILHQGQEAILQRYRMKGDHLRVYLHYLPSYYHLHVHFTALGFEAPGSGVERAHLLAEVIENLECDPRHYQQRMLTFALRADDPLLKLLQEAQQS

SEQ ID NO: 11 - DNA, 1508 bp, Artificial Sequence; identical to SEQ ID NO: 3
  mRNA in wild-type and C>T Exon 6 Thr316Met missense mutation

SEQ ID NO: 12 - DNA, 1508 bp, Artificial Sequence
  mRNA in wild-type and C>T Exon 6 Thr316Met missense mutation
gggcgtcttcggctcccaccccggggcgtgggcctgggggtccctcttcactgcgcagccacctctagcccgccgctttcgggactctcagccttcttttcccttccctgagggccggccggcaccacaacggggccaaaggcagtaactcggttgaaaggcgcgccctgggacaggtcagcgggctccgccatgcgcggcacgcatcccgcaggagcgggtgccagagcagggtaggccgggagttagcagcccagccaggccgctggactcgggctaggggcggggccagcgcagggggcgcaggcgcacaccgcctccgcggcagcatggcggacgcagctcctcaactaggcaagaggaagcgcgaattggacgtggaggaggcccacgccgccagcacagaggaaaaggaggcaggagttggaaatggtacctgtgctcctgtccgcttaccgttctccggcttcagactgcagaaggtgctgagggagtctgcgcgggacaaaatcattttcctacacgggaaggtgaatgaggcctctggggatggggatggagaggatgccgttgtgatcctggagaagacgccatttcaggtggaacaggtggctcagctcctgacgggcagccctgagctccaattgcagttctccaatgatatctacagcacctatcacttgttccctccaagacaactgaatgatgtaaagacgaccgtggtttaccctgccacagagaaacacctgcagaagtacctgcgccaggacctccgcctgatccgagagacgggagatgactacaggaatattactttaccccacctggagtcccagagcctcagcatccagtgggtgtataacattctcgacaagaaggctgaagcggaccggattgttttcgagaacccagatccctctgatggttttgtcctcatccctgaccttaagtggaaccaacagcagctcgatgacttgtacttgatcgccatctgccatcgccggggcatcagatccctacgcgaccttactccggagcacttgccgctgctcaggaacatcctccaccaggggcaggaggccatcctgcagcgctaccggatgaagggagaccatctgcgagtatacctgcactacctgccctcctactaccacctgcatgtgcacttcaccgccctgggcttcgaggcccccggctcaggcgtggagcgggcccacctgctggctgaggtgatcgagaacttggagtgtgaccctaggcactaccagcagcgcacgctcaccttcgccctcagggctgacgaccccctgctcaagctcttgcaggaggctcagcaaagctgaattaactcaggcagaagagcacagatgtgtgggattgggggaggagtggggacaagattttttatctccaagtgaattttctaaaaatgtattttataccggcttattcctagtattgaataaactagcgggctcaaaaaaaaaaaaaaaaaaaaaaaaaaaaaa

SEQ ID NO: 13 - DNA, 45 bp, Artificial Sequence
  portion of mRNA in wild-type and G>A intron 4 mutation
ataaaggtttctggctggaatgtcctgatctctggccaccctgct

SEQ ID NO: 14 - protein, 15 aa, Artificial Sequence
  portion of mRNA in wild-type and G>A intron 4 mutation
IKVSGWNVLISGHPA

SEQ ID NO: 15 - protein, 60 aa, human
GDHLRVYLHYLPSYYHLHVHFTALGFEAPGSGVERAHLLAEVIENLECDPRHYQQRTLTF

SEQ ID NO: 16 - protein, 60 aa, mouse
GDRLRVYLHYLPSYYHLHVHFTALGFEAPGSGVERAHLLAQVIENLECDPKHYQQRTLTF

SEQ ID NO: 17 - protein, 60 aa, opossum
EDRLRVYLHYQPSYYHLHVHFTALGFEAPGSGVERAHLLADVIENLEYDPQYYQKRTLTF

SEQ ID NO: 18 - protein, 60 aa, chicken
GSQLRIYLHYQPSYYHLHVHFTALGYDAPGISAERAHLLEDVIDNLLLDAQYYQKRALTF

SEQ ID NO: 19 - protein, 60 aa, Xenopus
GNKLRIYLHYQPSYYHLHVHFTVLGHDAPGSSVERAHLLCDVIQNIESNPQYYKTRTLTF

SEQ ID NO: 20 - protein, 60 aa, zebrafish
PSKLRVYFHYQPSYYHLHVHFTAVEYNVPGCGVERAHLLSDVIQNLQLDSSYYHNRSLSF

SEQ ID NO: 21 - protein, 60 aa, Tetraodon
ASKLRVYLHYQPSYYHLHVHFTMLGYEAPGCGVERAHLLGDVIQNLQSNPDYYKSHTLYF

SEQ ID NO: 22 - protein, 60 aa, lancelet
PCKLRIYLHYQPSYYHLHVHFTHLNFDAPGSGVERAHLLTDVIEHLEMMTDYYSKKTLSF

SEQ ID NO: 23 - protein, 60 aa, fruitfly
PNQLRMYFHYQPSFYHLHVHINPVRNDAPGIWCEKSHMLDTVINNLELMPDYYQRATLPF

SEQ ID NO: 24 - protein, 60 aa, nematode
TDQIKCYFHYQPSFYHLHVHFINLKYDAPASTTMSAILLDDVINNLELNPEHYKKSTLTF

(19) United States
(12) Patent Application Publication
(10) Pub. No.: US 2015/0315640 A1
(43) Pub. Date: Nov. 5, 2015

(54) IDENTIFICATION OF THE DCPS GENE ON 11Q24.2, WHICH ENCODES THE HUMAN DECAPPING ENZYME SCAVENGER, IN NON-SYNDROMIC AUTOSOMAL RECESSIVE MENTAL RETARDATION, DIAGNOSTIC PROBES THEREOF AND METHODS OF IDENTIFYING SUBJECTS WITH SAME

(71) Applicant: CENTRE FOR ADDICTION AND MENTAL HEALTH, Toronto (CA)

(72) Inventor: John B. Vincent, Toronto (CA)

(73) Assignee: Centre for Addiction and Mental Health, Toronto, Ontario (CA)

(21) Appl. No.: 14/441,160

(22) PCT Filed: Nov. 7, 2013
(86) PCT No.: PCT/CA2013/000945

§ 371 Date: May 6, 2015

(30) Foreign Application Priority Data
Nov. 7, 2012 (CA) 2794804

(51) Int. Cl. C12Q 1/68; G01N 33/573; C12N 9/14

(52) U.S. Cl. CPC C12Q 1/6883; C12N 9/14; G01N 33/573; C12Q 2600/156; C12Q 2600/16; C12Y 306/01059; G01N 2333/914; G01N 2800/28

(57) ABSTRACT

Provided herein is a DCPS nucleotide sequence on 11q24.2, which encodes the human decapping enzyme scavenger, associated with non-syndromic autosomal recessive mental retardation, diagnostic probes thereof, mutant proteins encoded thereby and methods of identifying subjects with same.

### FIELD OF INVENTION

The present invention relates to nucleotide sequences involved with mental retardation, diagnostic probes thereof and methods for identifying subjects with same. In particular, the present invention relates to identification of DCPS gene mutations related to ARMR.

### BACKGROUND OF THE INVENTION

Mental retardation (MR) is a devastating neurodevelopmental disorder with serious impact on the affected individuals and their families, as well as on health and social services. It is believed to occur with a prevalence of ˜2% within the population, and is frequently the result of genetic aberrations. MR may present as the sole clinical feature (non-syndromic), or may be present with additional clinical or dysmorphological features (syndromic). MR is significantly more frequent in males than in females, and it had been assumed that ˜25% of severe cases were X-linked, however a recent review suggests that X-linked mutations contribute to no more than 10% of cases (Ropers & Hamel, 2005). Little, however, is currently known about autosomal non-syndromic forms of MR. Autosomal recessive forms of non-syndromic MR (NS-ARMR) are believed to be more common, yet few genes have been identified so far.

### SUMMARY OF THE INVENTION

In an embodiment of the present invention, there is provided herein a nucleotide sequence comprising a G>A-intron 4-splice site mutation as defined herein, a nucleotide sequence complementary thereto, and a nucleotide sequence capable of hybridizing thereto but not a second nucleotide sequence having the wild type sequence.

In another embodiment, there is provided a nucleotide sequence comprising C>T-Exon6-Thr316Met mutation as defined herein, a nucleotide sequence complimentary thereto, a nucleotide sequence that is capable of hybridizing thereto, or a nucleotide sequence that is capable of hybridizing thereto but not a second nucleotide sequence encoding the Thr316 wild type of the sequence.

In still another embodiment of the present invention, there is provided a mutant DCPS protein as defined herein.

In a further embodiment, there is provided herein a nucleotide sequence probe or combination of probes that can be used to identify a subject with mental retardation as a result of genetic aberration in the DCPS protein or any other nucleotide sequence as described herein.

In a embodiment of the present invention, there is provided a method of screening or diagnosing a subject to identify any nucleotide sequences described herein associated with mental retardation or any protein sequences herein that are associated with mental retardation.

In a further embodiment, there is provided a kit comprising any nucleotide sequence or protein sequence described herein.

In yet another embodiment, there is provided a nucleotide sequence described herein which is 5, 6, 7, 8, 9, 10, 11, 12, 13, 14, 15, 16, 17, 18, 19, 20, 21, 22, 23, 24, 25, 26, 27, 28, 29, 30 or more nucleotides in length.

In a further embodiment, there is provided a nucleotide sequence that is at least 80% identical to any nucleotide sequence described herein, more preferably 81, 82, 83, 84, 85, 86, 87, 88, 89, 90, 91, 92, 93, 94, 95, 96, 97, 98, 99 or 100% identical thereto.

In an additional embodiment, there is provided a nucleic acid according to any one of SEQ ID Nos. 2 (SNP), 4 (SNP), 8 (SNP), or 12 (SNP). In a further embodiment, there is provided a fragment of a nucleic acid according to the embodiment above. In an additional embodiment, there is provided a nucleic acid substantially complementary to a nucleic acid according to the embodiments above. In a further embodiment, there is provided a cDNA nucleic acid corresponding to mRNA resulting from a DCPS gene comprising a SNP. In an additional embodiment, there is provided a nucleic acid with 70-100% sequence identity to a nucleic acid according to a nucleic acid described herein.

In an additional embodiment, there is provided a composition comprising one or more nucleic acids according to any embodiment described herein, and optionally one or more primers, polymerases, restriction enzymes, polymerases, and/or antibodies according to any one of the embodiments described herein or as known to one skilled in the art.

In a further embodiment, there is provided a protein according to any one of SEQ ID Nos. 6 (SNP) or 10 (SNP). In an additional embodiment, there is provided a fragment, truncated version, or mutated version of a DCPS protein according to any one of the sequences provided above or herein.

In an additional embodiment, there is provided a protein with 70-100% sequence identity to a protein according to the two embodiments described above, or any protein sequence described herein.

In an embodiment of the present invention, there is provided one or more nucleic acid primers or probes, optionally comprising a label or tag, capable of hybridizing to a nucleic acid sequence according to any of the sequences described above, a corresponding cDNA, or a corresponding complementary sequence.

In a further embodiment, there is provided one or more nucleic acid primers or probes capable of detecting a G>A intron 4 splice site mutation SNP in a DCPS gene.

In a further embodiment, there is provided one or more nucleic acid primers or probes capable of detecting a 45 nt insertion in DCPS mRNA resulting from a G>A intron 4 splice site mutation SNP. In an additional embodiment, there is provided one or more nucleic acid primers or probes capable of detecting a C>T Exon 6 Thr316Met missense mutation SNP in a DCPS gene or a resulting DCPS mRNA.

In a further embodiment of the present invention, there is contemplated a restriction enzyme capable of detecting the presence of a G>A intron 4 splice site mutation SNP in a nucleic acid as described herein. In another embodiment of the present invention, there is provided a restriction enzyme capable of detecting the presence of a 45 nt insertion in DCPS mRNA resulting from a G>A intron 4 splice site mutation SNP. In an additional embodiment, there is provided a restriction enzyme capable of detecting the presence of a C>T Exon 6 Thr316Met missense mutation SNP in a DCPS gene or a resulting DCPS mRNA.

In another embodiment of the present invention, there is provided an antibody for detecting a mutant DCPS protein resulting from a G>A intron 4 splice site mutation SNP, wherein said antibody optionally detects some or all of the amino acid sequence IKVSGWNVLISGHPA. In a further embodiment, there is provided an antibody for detecting a mutant DCPS protein resulting from a C>T Exon 6 Thr316Met missense mutation SNP.

In one embodiment of the present invention, there is provided a kit for detecting a DCPS G>A intron 4 splice site mutation SNP, said kit comprising:
reagents (primers, polymerase, NTP5) and instructions for PCR amplification and sequencing; or
microarrays for SNP detection; or
one or more restriction enzymes; or
one or more nucleic acid probe sequences for SNP detection.

In another embodiment of the present invention, there is provided a kit for detecting a DCPS C>T Exon 6 Thr316Met missense mutation SNP, said kit comprising:
reagents (primers, polymerase, NTPs) and instructions for PCR amplification and sequencing; or
microarrays for SNP detection; or
one or more restriction enzymes; or
one or more nucleic acid probe sequences for SNP detection.

In a further embodiment of the present invention, there is provided a kit for detecting a mutant mRNA resulting from a DCPS G>A intron 4 splice site mutation SNP, said kit comprising:
reagents (primers, polymerase, NTPs) and instructions for PCR amplification and sequencing; or
microarrays for SNP detection; or
one or more restriction enzymes; or
one or more nucleic acid probe sequences for SNP detection.

In an additional embodiment of the present invention, there is provided a kit for detecting a mutant mRNA resulting from a DCPS C>T Exon 6 Thr316Met missense mutation SNP, said kit comprising:
reagents (primers, polymerase, NTPs) and instructions for PCR amplification and sequencing; or
microarrays for SNP detection; or
one or more restriction enzymes; or
one or more nucleic acid probe sequences for SNP detection.

In a further embodiment of the present invention, there is provided a kit for detecting a mutant protein resulting from a DCPS G>A intron 4 splice site mutation SNP, said kit comprising:
one or more antibodies for detecting a mutant DCPS protein.

In another embodiment of the present invention, there is provided a kit for detecting a mutant protein resulting from a DCPS C>T Exon 6 Thr316Met missense mutation SNP, said kit comprising:
one or more antibodies for detecting a mutant DCPS protein.

In an embodiment of the present invention, there is provided a method of detecting a DCPS G>A intron 4 splice site mutation SNP, said method comprising the steps of:
amplifying a nucleic acid sequence comprising the DCPS G>A intron 4 splice site mutation SNP site;
identifying the presence or absence of a G>A mutation at the SNP site; or comprising the steps of:
contacting a SNP-detecting microarray with a sample;
identifying the presence or absence of a G>A mutation at the SNP site; or comprising the steps of:
contacting a sample with an SNP-detecting restriction enzyme;
identifying the presence or absence of a G>A mutation at the SNP site based on restriction enzyme cleavage patterns;
   or comprising the steps of:
contacting a sample with an SNP-detecting nucleic acid probe;
identifying the presence or absence of a G>A mutation at the SNP site based on probe interactions with nucleic acids in the sample.

In a further embodiment, there is provided a method of detecting a DCPS C>T Exon 6 Thr316Met missense mutation SNP, said method comprising the steps of:
amplifying a nucleic acid sequence comprising the C>T Exon 6 Thr316Met missense mutation SNP site;
identifying the presence or absence of a C>T mutation at the SNP site; or comprising the steps of:
contacting a SNP-detecting microarray with a sample;
identifying the presence or absence of a C>T mutation at the SNP site;
   or comprising the steps of:
contacting a sample with an SNP-detecting restriction enzyme;
identifying the presence or absence of a C>T mutation at the SNP site based on restriction enzyme cleavage patterns;
   or comprising the steps of:
contacting a sample with an SNP-detecting nucleic acid probe;
identifying the presence or absence of a C>T mutation at the SNP site based on probe interactions with nucleic acids in the sample.

In an additional embodiment of the present invention, there is provided a method of detecting a mutant mRNA resulting from a DCPS G>A intron 4 splice site mutation SNP, said method comprising the steps of:
producing a cDNA from an mRNA resulting from a DCPS G>A intron 4 splice site mutation SNP;
amplifying the cDNA;
identifying the presence or absence of a 15 nt insertion mutation;
   or comprising the steps of:
contacting a SNP-detecting microarray with a sample;
identifying the presence or absence of a 15 nt insertion mutation;
   or comprising the steps of:
contacting a sample with an SNP-detecting restriction enzyme;
identifying the presence or absence of a 15 nt insertion mutation based on restriction enzyme cleavage patterns;
   or comprising the steps of:
contacting a sample with 15 nt insertion mutation-detecting nucleic acid probe;
identifying the presence or absence of a 15 nt insertion mutation based on probe interactions with nucleic acids in the sample;
   or comprising the steps of:
resolving a sample using a gel electrophoresis-based nucleic acid analysis technique;
identifying the presence or absence of a 15 nt insertion DCPS mRNA mutant based on differential mobility.

In an additional embodiment of the present invention, there is provided a method of detecting a mutant mRNA resulting from a DCPS C>T Exon 6 Thr316Met missense mutation SNP, said method comprising the steps of
producing a cDNA from an mRNA resulting from a DCPS C>T Exon 6 Thr316Met missense mutation SNP;
amplifying the cDNA;
identifying the presence or absence of a Thr316Met missense mutation;
   or comprising the steps of:
contacting a SNP-detecting microarray with a sample;
identifying the presence or absence of a Thr316Met missense mutation;
   or comprising the steps of:
contacting a sample with an SNP-detecting restriction enzyme;
identifying the presence or absence of a Thr316Met missense mutation based on restriction enzyme cleavage patterns;
   or comprising the steps of:
contacting a sample with a Thr316Met missense mutation-detecting nucleic acid probe;
identifying the presence or absence of a Thr316Met missense mutation based on probe interactions with nucleic acids in the sample.

In another embodiment of the present invention, there is provided a method of detecting a mutant protein resulting from a DCPS G>A intron 4 splice site mutation SNP, said method comprising the steps of:
contacting a sample with an antibody capable of detecting a protein mutation resulting from a DCPS G>A intron 4 splice site mutation SNP;
identifying the presence or absence of a DCPS G>A intron 4 splice site mutation SNP based on antibody interaction with protein in the sample;
   or comprising the steps of
analyzing a sample using mass spectrometry;
identifying the presence or absence of G>A intron 4 splice site mutation DCPS protein, or fragments generated from said mutant protein, in said sample;
identifying the presence or absence of a DCPS G>A intron 4 splice site mutation SNP based on the presence or absence of G>A intron 4 splice site mutant protein or protein fragments.

In a further embodiment, there is provided a method of detecting a mutant protein resulting from a DCPS C>T Exon 6 Thr316Met missense mutation SNP, said method comprising the steps of:
contacting a sample with an antibody capable of detecting a protein mutation resulting from a DCPS C>T Exon 6 Thr316Met missense mutation SNP;
identifying the presence or absence of a DCPS C>T Exon 6 Thr316Met missense mutation SNP based on antibody interaction with protein in the sample;
   or comprising the steps of:
analyzing a sample using mass spectrometry;
identifying the presence or absence of C>T Exon 6 Thr316Met missense mutation DCPS protein, or fragments generated from said mutant protein, in said sample;
identifying the presence or absence of a DCPS C>T Exon 6 Thr316Met missense mutation SNP based on the presence or absence of C>T Exon 6 Thr316Met missense mutant protein or protein fragments.

In an additional embodiment of the present invention, there is provided a method of amplifying a nucleic acid related wild-type or mutant DCPS, said method comprising the steps of:
contacting a sample with one or more PCR primers, polymerase enzyme, and nucleotide triphosphates;
using PCR to amplify the nucleic acid;
   or comprising the steps of:
generating an expression vector or plasmid coding for the nucleic acid to be amplified;
expressing the vector or plasmid to produce the encoded nucleic acid sequence.

In a further embodiment, there is provided a method of producing an antibody capable of recognizing a wild-type or mutant DCPS protein as described herein, comprising the steps of:
generating a sample of said protein,
using said protein sample to generate monoclonal or polyclonal antibodies using known techniques, such as hybridoma cell production or inoculation of a suitable mammal with an antigen comprising said protein.

In an additional embodiment of the present invention, there is provided a method for screening or diagnosing a subject for a NSARMR-linked DCPS SNP mutation, said method comprising the steps of
analyzing a sample from the subject to determine the presence or absence of a S G>A intron 4 splice site mutation SNP;
analyzing a sample from the subject to determine the presence or absence of a C>T Exon 6 Thr316Met missense SNP mutation;
   wherein the presence of one or both SNP mutations indicates an NSARMR-linked DCPS gene mutation.

In a further embodiment of the present invention, there is provided a method for screening or diagnosing a subject for a NSARMR-linked DCPS SNP mutation, said method comprising the steps of:
analyzing a sample from the subject to determine the presence or absence of a DCPS G>A intron 4 splice site mutation SNP; and,
if a DCPS G>A intron 4 splice site mutation SNP is present, further analyzing to determine the presence or absence of a C>T Exon 6 Thr316Met missense SNP mutation;
wherein the presence of one or both SNP mutations indicates an NSARMR-linked DCPS gene mutation.

A method for screening or diagnosing a subject for a NSARMR-linked DCPS SNP mutation, said method comprising the steps of:
analyzing a sample from the subject to determine the presence or absence of a DCPS C>T Exon 6 Thr316Met missense SNP mutation SNP is present; and,
if a DCPS C>T Exon 6 Thr316Met missense SNP mutation SNP is present, further analyzing to determine the presence or absence of a DCPS G>A intron 4 splice site mutation SNP;
   wherein the presence of one or both SNP mutations indicates an NSARMR-linked DCPS gene mutation.

According to an embodiment of the present invention, there is provided a Decapping Enzyme Scavenger nucleotide sequence associated with Non-Syndromic Autosomal Recessive Mental Retardation, the nucleotide sequence comprising:

a) at least 7 consecutive nucleotides of SEQ ID NO:2 and comprising “A” at position 51, or a sequence complementary thereto;
b) at least 7 consecutive nucleotides of SEQ ID NO:4 and further comprising ATAAAGGTTTCTGGCTGGAATGTCCTGATCTCTGGCCACCCTGCT defined by SEQ ID NO:13, or a fragment thereof, or a nucleotide sequence that is complementary thereto;
c) at least 7 consecutive nucleotides of SEQ ID NO:8 and comprising T at position 271, or a sequence complementary thereto;
d) at least 7 consecutive nucleotides of SEQ ID NO:12 and further comprising Tat position 947, or a sequence complementary thereto, or;
e) at least 80% identity to the nucleotide sequence defined in any one of a)-d).

In a preferred embodiment, the nucleotide sequence as described above comprises at least 7 consecutive nucleotides but in other embodiments may comprise 8, 9, 10, 11, 12, 13, 14, 15, 16, 17, 18, 19, 20, 21, 22, 23, 24, 25 or more. The upper end size may comprise any value for example 100, 200, 300, 400, 500, 600, 700, 800, 900, 1000, 2000, 3000, 4000, 5000 or more nucleotides.

In a further embodiment, the nucleotide sequence the nucleotide sequence as described above and herein comprises at least 7 consecutive nucleotides of SEQ ID NO:4 and further comprising ATAAAGGTTTCTGGCTGGAATGTCCTGATCTCTGGCCACCCTGCT defined by SEQ ID NO:13, or a fragment thereof which is at least 1 continuous nucleotide, more preferably still 2, 3, 4, 5, 6, 7, 8, 9, 10, 11, 12, 13, 14, etc continuous nucleotides thereof up to the full sequence of SEQ ID NO:13.

The present invention also contemplates a nucleotide sequence as described herein which is labeled, for example, but not wishing to be limiting, by a fluorescent tag or label, a radioactive group or the like. Any label which permits the nucleotide sequence to be identified over other nucleotide sequences is contemplated.

Also provided herein is a composition comprising the nucleotide sequence as described above further comprising one or more primers that bind to the nucleotide sequence, a thermophilic DNA polymerase, restriction enzyme or any combination thereof. In a further embodiment, the composition is a nucleotide sequence amplification mixture.

Also provided by the present invention is a vector comprising the nucleotide sequence as described herein.

In a further embodiment, there is provided a polypeptide defined by SEQ ID NO:6 or SEQ ID NO:10.

Also provided by the present invention is a polypeptide defined as comprising 70% to 100% identity to a) the polypeptide defined above and herein, b) SEQ ID NO:6 and comprising a 15 amino acid insertion defined by IKVSGWNVLISGHPA (SEQ ID NO:14) or c) SEQ ID NO:10 and comprising Met at position 316.

The present invention also contemplates an isolated nucleic acid sequence encoding the polypeptide as described above.

Also contemplated is an antibody that binds to the polypeptide sequence defined by IKVSGWNVLISGHPA (SEQ ID NO:14) or SEQ ID NO:10 when comprising Met at position 316.

The present invention also provides a kit comprising one or more nucleotide sequences defined herein and optionally any one or combination of a polypeptide, vector, composition or antibody as described herein and optionally further comprising one or more buffers, primers, restriction enzymes, dNTPs, microarrays, gene chips, assay plates, multi-well dishes, glass substrates, purification resins or beads or any combination thereof, wherein the nucleotide sequence, polypeptide, vector, composition or antibody is optionally physically associated with or attached to the buffer, primer, restriction enzyme, dNTP, microarray, gene chip, assay plate, multi-well dish, glass substrate, purification resin or bead.

The present invention also provides a method of detecting or screening a subject for a nucleotide sequence or protein associated with mental retardation comprising,

a) obtaining a biological sample from the subject, the sample comprising genomic DNA, mRNA or protein from the subject;
b) identifying the presence or absence the nucleotide sequence as defined herein or the protein as defined herein, wherein the presence of the nucleotide sequence or protein is indicative that the subject has or is at risk for developing cognitive deficits associated with mental retardation or is a carrier for one or more genes associated with mental retardation. In a further embodiment, the step of identifying is performed by microarray analysis, restriction analysis, probe hybridization, nucleotide sequence amplification, PCR, electrophoretic-based nucleic acid analysis, ELISA, DNA sequencing, protein sequencing, antibody binding analysis, mass spectrometry or any combination thereof.

Other embodiments that are important are also disclosed throughout.

### BRIEF DESCRIPTION OF THE DRAWINGS

These and other features of the invention will become more apparent from the following description in which reference is made to the appended drawings wherein:

FIG. 1 (upper) shows the Pedigree of a family from Pakistan. FIG. 1 (middle) shows HomozygosityMapper analysis (Seelow et al, 2009) for microarray SNP data: Genome-wide. Significant regions of HBD are seen on 11q and other loci 14q and 17q were excluded because one or other unaffected siblings were also homozygous at these loci. FIG. 1 (lower) shows IGV Snapshot from chr11:126208295 G>A showing substitution at splice donor site reveal by NGS data analysis.

FIG. 2 (upper) shows a chromatogram of Father (IV-5), Mother (IV-6) and affected daughter (V-7) showing segregation of compound heterozygous mutation responsible for disease phenotypes. In FIG. 2 (lower) there is shown a diagrammatic illustration of compound heterozygous mutations in V-7 individual.

FIG. 3 shows predicted structures for wild type and splice site mutant DCPS enzyme.

FIG. 4 shows CLUSTALW alignment for DCPS protein at Thr316Met site (highlighted, underlined) across multiple species, plus POLYPHEN2 prediction of the effect of the Thr316Met amino acid substitution.

FIG. 5 shows the sequences of chromosome 11 between positions 126208245 and 126208344 in both wild type (top) and G>A-intron4-splice site mutation (bottom) alleles. The G>A mutation is highlighted, underlined.

FIG. 6 shows the sequences of mRNA produced from wild-type (top) and G>A-intron4-splice site mutation (bottom) genes. The sequence insertion resulting from the SNP mutation is highlighted, underlined.

FIG. 7 shows the sequences of protein produced from wild-type (top) and G>A-intron4-splice site mutation (bottom) genes. The amino acid insertion resulting from the SNP mutation is highlighted, underlined.

FIG. 8 shows the sequences of chromosome 11 between positions 126215171 and 126215460 in both wild type (top) and C>T-Exon6-Thr316Met mutation (bottom) alleles. The C>T mutation is highlighted, underlined.

FIG. 9 shows the sequences of protein produced from wild-type (top) and C>T-Exon6-Thr316Met mutation (bottom) genes. The missense Thr316Met mutation is highlighted, underlined.

FIG. 10 shows the sequences of mRNA produced from wild-type (top) and C>T-Exon6-Thr316Met mutation (bottom) genes. The SNP site is highlighted, underlined.

FIG. 11 upper shows the nucleotide sequence of SEQ ID NO:13 demonstrating the underlined nucleotide sequence indicated in SEQ ID NO:4. FIG. 11 lower shows SEQ ID NO:14 which illustrates the additional amino acid sequence that is added to the protein defined by SEQ ID NO:6.

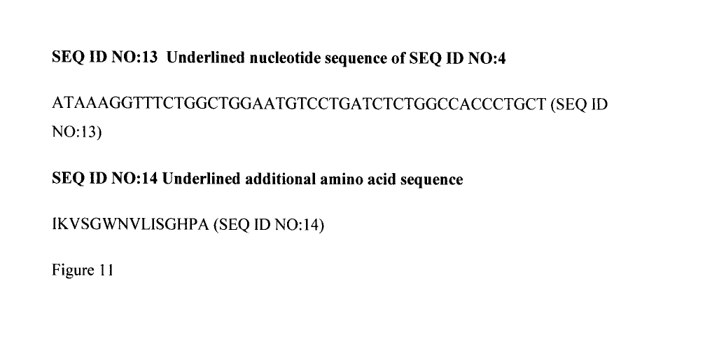

FIG. 12 shows the activity of wild-type, G>A-intron4-splice site mutant, and C>T-Exon6-Thr316Met mutant DCPS enzymes in decapping assays. DcpS catalyzes the hydrolysis of cap structure. Decapping assays were carried out with the indicated amounts of DcpS at the top of picture. 32P-labeled methylated cap structure (m7Gpppp) is used as substrate where red colour “p” in m7GpppG represented labelled phosphate and m7Gp as product of DcpS catalyzed reaction. The quantitation for the decapping efficiency of each protein is presented as the percentage decapping using ImageQuant 5.2 software at the bottom of picture.

FIG. 13 shows decapping assays of wild type and mutant DCPS from whole cell extract. The quantitation for the decapping efficiency of each protein is presented as the percentage decapping using ImageQuant 5.2 software. The standard m7Gppp and positive control are shown at left. rDcpS are wild type control second from left. 32P-labeled methylated cap structure (m7Gpppp) is used as substrate where red colour “p” in m7GpppG represented labelled phosphate and m7Gp as product of DcpS catalyzed reaction.

FIG. 14 shows Western blot analysis showing expression concentration of DcpS in lymphoblast cell lines of generated from patient with homozygous 15 Amino Acid insertion in lane 1 and 4 and heterozygous individuals of Family A in lane 2 and 3. 293T was used for positive control and GAPDH as loading control.

### DETAILED DESCRIPTION

The following description is of a preferred embodiment.

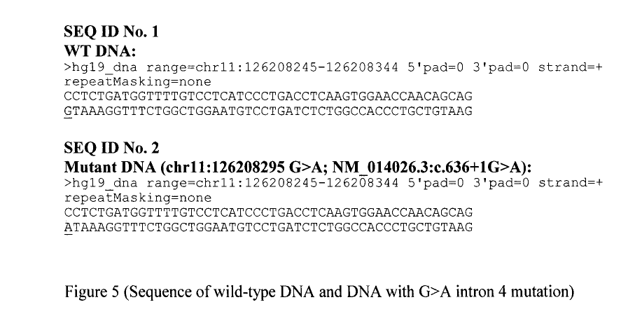

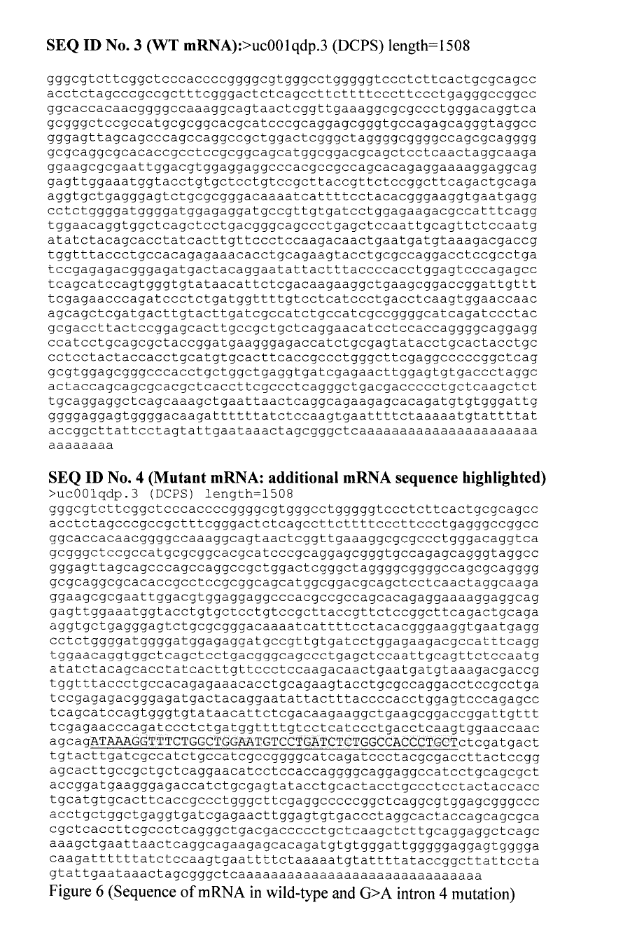

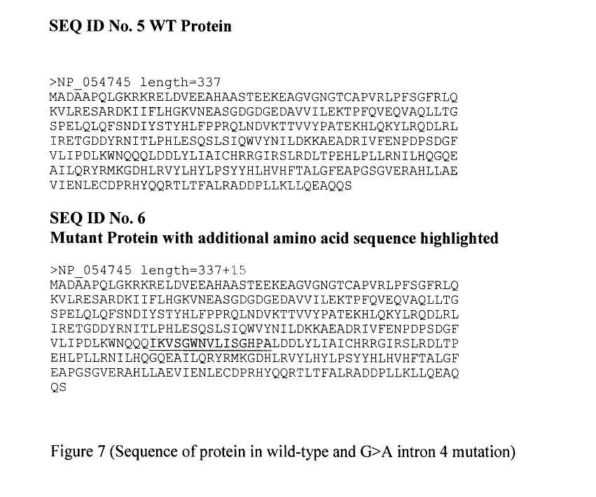

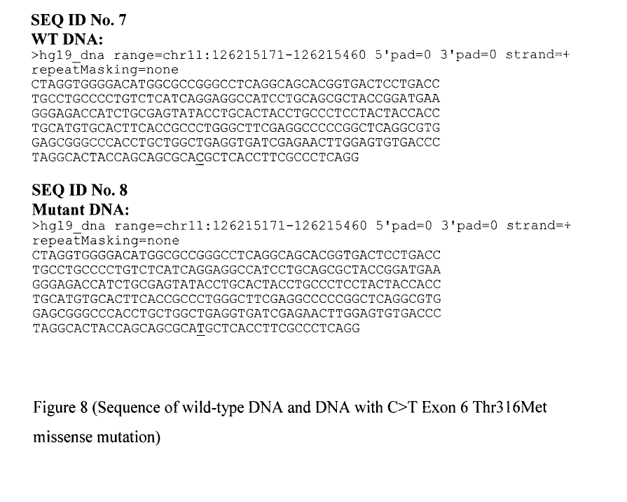

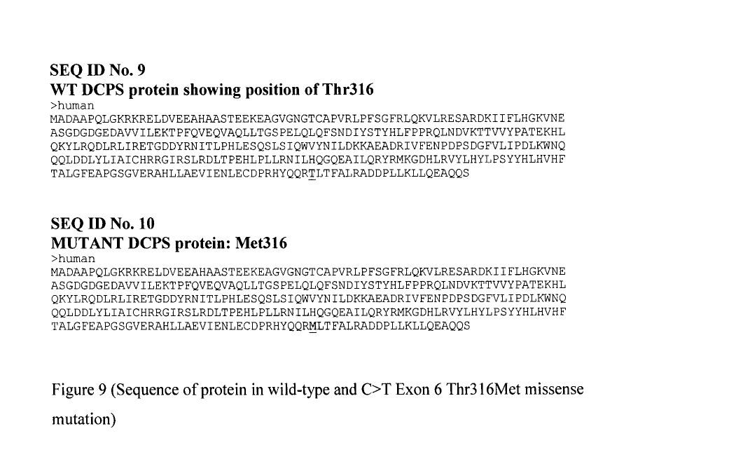

We have used homozygosity mapping together with whole exome sequencing to search for the gene responsible for NS-ARMR in a large Pakistani pedigree. Using Affymetrix 500K SNP microarrays, we identified a 11 Mb region on 11q24.1-q25. with a continuous run of 1269 SNPs homozygous common among two affecteds in the family (homozygous-by-descent, or HBD). Two other HBD loci, on 14q11.2 and 17q24.2-q24.3, were excluded after genotyping highly polymorphic microsatellite markers across the region in all family members. Thus the 11q locus was identified as harbouring a gene for NS-ARMR. Whole exome sequencing for one of the affected individuals was performed. We identified a homozygous G>A base substitution at the splice donor +1 site in intron 4 (shown in FIG. 5). By sequencing mRNA from affected individuals, we have shown that a cryptic splice donor 44 nucleotides downstream of this is used, and splicing with exon 4 maintains the frame, leading to the insertion of 15 amino acids (shown in FIGS. 6 and 7). This additional protein sequence is predicted to disrupt crucial protein function in the decapping of mRNAs. In silico analysis shows a significant predicted effect on the protein folding. In a third affected individual, HBD at this region was not seen, however sequencing confirmed her not only to be heterozygous for the intron 4 splice donor mutation, but to be heterozygous for a missense mutation Thr316Met (shown in FIGS. 8, 9, and 10). We screened a population of healthy Pakistani controls, and neither mutation was present among the 200 subjects tested. Furthermore, neither mutation has been identified by large scale whole exome or genome sequencing projects (1000 Genomes and NHLBI Exome Sequencing Project).

We have identified the DCPS gene as a cause for autosomal recessive mental retardation (ARMR). Mutations within this gene have not been associated with any phenotype previously. According to an embodiment of the present invention, the information provided here allows for direct diagnosis of mental retardation individuals on the basis of genetic mutation screening of DCPS. Furthermore, the present invention also provides for genetic diagnostics for mental retardation and assists with the impact on genetic counseling for families with mental retardation. The present invention also provides possible therapeutic intervention for mental retardation and for related phenotypes, including autism, through targeting expression of genes specific to the biochemical pathway for the DCPS protein.

According to one embodiment of the present invention, there is provided a method for detecting one or both of the G>A-intron4-splice site and C>T-Exon6-Thr316Met SNP mutations in a subject or in a sample. These DCPS SNP sites are illustrated in FIGS. 5 and 8, SEQ ID Nos. 1, 2, 7, and 8. It should be noted that the illustrated sequences may also be considered in the context of the larger nucleotide sequence, for example, but not limited to a vector, cloning vector, gene or chromosome.

According to another embodiment of the present invention, there is provided a method for detecting in a subject or sample one or both of the mutated mRNA sequences produced by one or both of the G>A-intron4-splice site and C>T-Exon6-Thr316Met SNP mutations.

According to another embodiment, there is provided a means for detecting a nucleic acid having sequence according to all or a portion of SEQ ID Nos. 2, 4, 8, and/or 12, or a nucleic acid complementary to any of these sequences, in a subject or in a sample.

As will be appreciated by a person skilled in the art, there are several well-known methods for detecting SNPs, all of which may be contemplated in the embodiments described herein. Non-limiting examples include known sequencing techniques which may or may not involve a PCR amplification step, microarray-based methods, exome sequencing methods, restriction enzyme-based methods, FISH-based methods, DASH-based methods, molecular beacon methods, and other methods known in the art.

In another embodiment according to the present invention, there is provided a method for detecting one or both of the mutant proteins resulting from the G>A-intron4-splice site and C>T-Exon6-Thr316Met SNP mutations. The G>A-intron4-splice site SNP mutant protein has an additional 15 nucleotides not found in the wild-type protein (FIG. 7), and the C>T-Exon6-Thr316Met SNP mutant protein has a Met in place of Thr in the wild-type protein. As will be appreciated by a person skilled in the art, there are several well-known methods for detecting the presence of these mutant proteins and distinguishing them from wild-type protein. Non-limiting examples include mass-spectrometry-based methods, and antibody-based detection methods.

According to an aspect of the present invention, there is provided a method of amplifying, for example by PCR, a nucleic acid sequence containing position 126208245 of SEQ ID NOs:1 or 2 (FIG. 5) or position 126215431 of SEQ ID Nos. 7 or 8 using a first primer that binds upstream of said position and a second primer that binds downstream of said position; detecting the presence of a A nucleotide at position 126208245 of SEQ ID NOs:1 or 2 (FIG. 5) and/or a T nucleotide at position 126215431 of SEQ ID Nos. 7 or 8; and determining the genotype of the human subject at positions 126208245 and/or 126215431.

According to another aspect of the present invention, amplification of a nucleic acid sequence containing position 126208245 of SEQ ID NOs:1 or 2 (FIG. 5) or position 126215431 of SEQ ID Nos. 7 or 8 may be achieved using any of the techniques known in the art, including but not limited to expression from expression vectors or plasmids, and rolling circle replication-based amplification methods.

According to a further aspect of the present invention, there is provided a method substantially similar to the method immediately above, wherein the positions complementary to the positions identified above are interrogated in sequences complementary to those identified above.

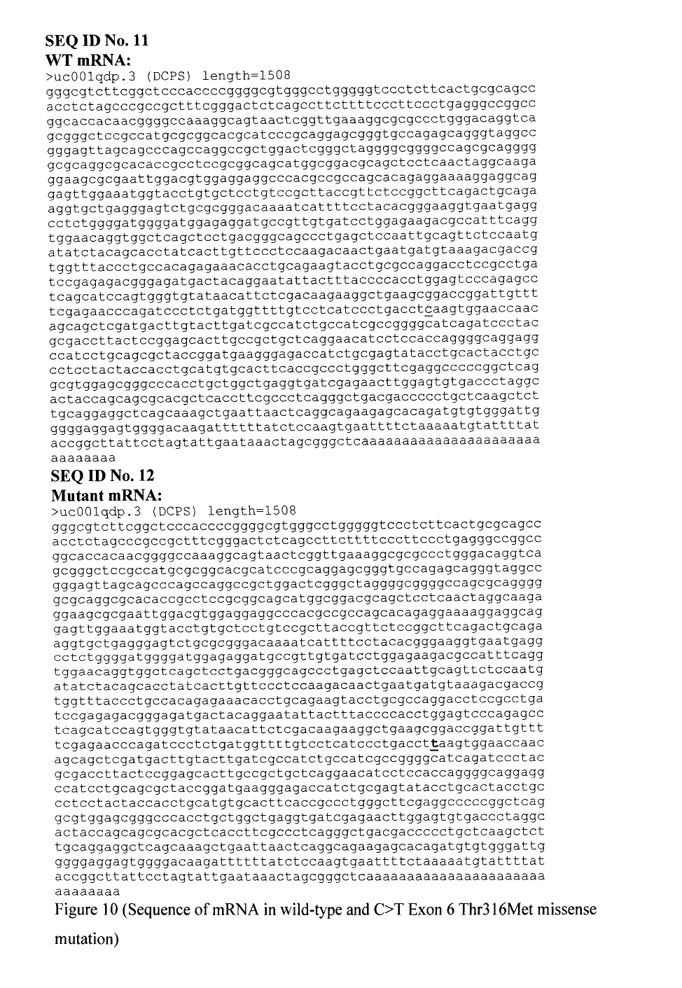

According to an aspect of the present invention, there is provided a method of amplifying, for example by PCR, a nucleic acid sequence of SEQ ID NOs:3 or 4 (FIG. 6) or SEQ ID Nos. 11 or 12 or fragment thereof using a first primer that binds upstream of the site of the 45 nt insertion introduced by the G>A-intron4-splice site SNP in SEQ ID NOs 3 or 4, or upstream of position 947 in SEQ ID NOs 11 or 12 and a second primer that binds downstream of said positions; detecting the presence of a 45 nt insertion in SEQ ID NOs:3 or 4 (FIG. 5) and/or a T nucleotide at position 947 of SEQ ID Nos. 11 or 12 (FIG. 10); and determining the genotype of the human subject at the G>A-intron4-splice site and/or C>T-Exon6-Thr316Met SNPs described herein.

According to an embodiment of the present invention, there is provided an amplification method substantially similar to that above, wherein an mRNA according to a nucleic acid sequence of SEQ ID NOs:3 or 4 (FIG. 6) or SEQ ID Nos. 11 or 12 or fragment thereof is converted to a corresponding cDNA prior for further amplification.

According to a further aspect of the present invention, there is provided a method substantially similar to the two methods immediately above, wherein the positions complementary to the positions identified above are interrogated in sequences complementary to those identified above.

According to a further aspect of the present invention, there is provided a method for identifying the presence of a G>A-intron4-splice site SNP mutation by analyzing a sample using gel electrophoresis techniques well-known in the art to identify the presence or absence of the 45 nt insertion in DCPS mRNA caused by the G>A-intron4-splice site SNP mutation.

The sample obtained from a subject may comprise any biological sample from which genomic DNA may be isolated, for example, but not to be limited to a tissue sample, a sample of saliva, a cheek swab sample, blood, or other biological fluids that contain genomic DNA. In a preferred embodiment, which is not meant to be limiting in any manner, the sample is a blood sample. In another embodiment, RNA or mRNA is isolated from the subject.

The method of obtaining and analyzing DNA or RNA is not critical to the present invention and any method or methods may be used (e.g. Ausubel, et al. (eds), 1989, Current Protocols in Molecular Biology, Green Publishing Associates, Inc., and John Wiley & Sons, Inc., New York, at p. 2.10.3, or Maniatis et al., in Molecular Cloning (A Laboratory Manual), Cold Spring Harbor Laboratory, 1982, p. 387-389). For example, which is not to be considered limiting in any manner, DNA may be extracted using a non-enzymatic high-salt procedure (Lahiri and Nurnberger 1991). Alternatively, the DNA may be analyzed in situ. RNA can isolated, for example, by phenol chloroform extraction and analyzed using RT-PCR.

Genotyping of the G>A-intron4-splice site SNP and C>T-Exon6-Thr316Met SNP as described herein may be performed by any method known in the art, for example PCR, sequencing, ligation chain reaction (LCR) or any other standard method known in the art that may be used to determine SNPs (single nucleic acid polymorphisms). In an embodiment, which is not meant to be limiting in any manner, amplifying the nucleic acid sequences containing the G>A-intron4-splice site SNP and/or C>T-Exon6-Thr316Met SNP and genotyping the same is performed by PCR analysis using appropriate primers, probes and PCR conditions.

In one embodiment, the step of amplifying the sequences containing the G>A-intron4-splice site SNP and/or the C>T-Exon6-Thr316Met SNP involves subjecting the nucleic acid sample to PCR, wherein the program for denaturing, annealing, amplifying is stored on a computer readable medium for execution by a microprocessor. The program causes a machine containing the samples to cycle through various temperatures for set periods of time. A similar or different machine comprising one or more programs may be employed to convert physical information, for example, but not limited to binding of nucleic acids or probes to target sequences, amplification or the like to a different state, such as electronic or otherwise, for example a signal that can be printed, displayed pictorially or digitized.

In a further embodiment, a restriction enzyme, preferably from a bacteria or virus, may be used to detect the presence of the G>A-intron4-splice site SNP and/or the C>T-Exon6-Thr316Met SNP. One or more restriction enzymes may recognize the consensus sequence around one or both SNPs, and differentially cleave or not cleave wild-type versus mutant sequence. As such, a restriction enzyme recognizing all or a fragment of one or more nucleic acids with sequence according to SEQ ID Nos. 1-4, 7, 8, 11, or 12, or the complementary wild-type or mutated sequence, can be used to easily determine the genotype of the subject. Other methods also may be used.

An apparatus, such as microarray or DNA chip, can be used to detect the presence or absence of the G>A-intron4-splice site SNP and/or the C>T-Exon6-Thr316Met SNP or any other nucleic acid which results in a mutated DCPS protein as described herein. In this case, but without wishing to be limiting in any manner, an oligonucleotide may be bound to a substrate, which is suitable for this type of application. In an embodiment the oligonucleotide preferably comprises a contiguous nucleic acid, for example, the sequence from one or more of SEQ ID NOs. 2, 4, 8, and 12 containing one or both SNPs described herein or a sequence substantially identical thereto. Another oligonucleotide can also be bound to the substrate. For example, but not wishing to be limiting, a nucleotide sequence comprising a complement of the nucleic acid sequences provided immediately above. In one embodiment the oligonucleotides are 7, 10, 12, 15, 16, 17, 19, 21, 23, 25 or more nucleotides in length. In another embodiment, the oligonucleotides are 60 nucleotides in length or more. Alternatively, the oligonucleotides may be defined by a range of any two of the values noted above or any two values therein between. A person skilled in the art will recognize that the length of the oligonucleotides can be altered based on the parameters of the assay. It is envisaged that the apparatus can contain other oligonucleotide sequences to confirm the subject's diagnosis or to test for the susceptibility of additional diseases or disorders, comorbid or otherwise.

The present invention also contemplates screening methods which identify and/or characterize the proteins as defined herein within biological samples from subjects. Such samples may or may not comprise DNA or RNA. For example, such screening or testing methods may employ immunological methods, for example, but not limited to antibody binding assays such as ELISAs or the like, protein sequencing, electrophoretic separations to identify the proteins as described above in a sample. As will be evident to a person of skill in the art, the screening methods allow for the differentiation of the proteins as defined herein from wild type proteins known in the art.

Also contemplated by the present invention is a nucleic acid comprising or consisting of a sequence selected from the group consisting of: a) a nucleic acid sequence comprising SEQ ID NOs. 1-4, 7, 8, 11, or 12; b) a complement of a nucleic acid sequence comprising SEQ ID NOs. 1-4, 7, 8, 11, or 12; c) a fragment of either a) or b); d) a nucleic acid sequence capable of hybridizing to any one of a), b) or c); and e) a nucleic acid sequence that exhibits greater than about 70% sequence identity with the nucleic acid defined in a), b) c) or d).

A nucleic acid sequence exhibiting at least 70% identity thereto is understood to include sequences that exhibit 70%, 75%, 80%, 85%, 90%, 91%, 92%, 93%, 94%, 95%, 96%, 97%, 98%, 99%, 99.9% or 100% identity, or an value therein between to SEQ ID NOs. 1-4, 7, 8, 11, or 12. Further, the nucleic acid may be defined as comprising a range of sequence identity as defined by any two of the values listed or any values therein between.

Any method known in the art may be used for determining the degree of identity between nucleic acid sequences. For example, but without wishing to be limiting, a sequence search method such as BLAST (Basic Local Alignment Search Tool: (Altschul S F, Gish W, Miller W, Myers E W, Lipman D J (1990) J Mol Biol 215: 403-410) can be used according to default parameters as described by Tatiana et al., FEMS Microbiol Lett. 174:247-250 (1990), or on the National Center for Biotechnology Information web page at ncbi.nlm.gov/BLAST/, for searching closely related sequences. BLAST is widely used in routine sequence alignment; modified BLAST algorithms such as Gapped BLAST, which allows gaps (either insertions or deletions) to be introduced into alignments, PSI-BLAST, a sensitive search for sequence homologs (Altschul et al., (1997) Nucleic Acid Res. 25:3389-3402); or FASTA, which is available on the world wide web at ExPASy (EMBL-European Bioinformatics Institute). Similar methods known in the art may be employed to compare DNA or RNA sequences to determine the degree of sequence identity.

Stringent hybridization conditions may be, for example but not limited to hybridization overnight (from about 16-20 hours) hybridization in 4×SSC at 65° C., followed by washing in 0.1×SSC at 65° C. for an hour, or 2 washes in 0.1×SSC at 65° C. each for 20 or 30 minutes. Alternatively, an exemplary stringent hybridization condition could be overnight (16-20 hours) in 50% formamide, 4×SSC at 42° C., followed by washing in 0.1×SSC at 65° C. for an hour, or 2 washes in 0.1×SSC at 65° C. each for 20 or 30 minutes, or overnight (16-20 hours); or hybridization in Church aqueous phosphate buffer (7% SDS; 0.5M NaPO4 buffer pH 7.2; 10 mM EDTA) at 65° C., with 2 washes either at 50° C. in 0.1×SSC, 0.1% SDS for 20 or 30 minutes each, or 2 washes at 65° C. in 2×SSC, 0.1% SDS for 20 or 30 minutes each for unique sequence regions.

Also contemplated by the present invention is a method comprising the steps of: isolating RNA from the subject; hybridizing an oligonucleotide comprising a contiguous nucleic acid capable of hybridizing to a nucleic acid of SEQ ID NOs. 4 or 12 but not to a nucleic acid of SEQ ID NOs. 3 and 11 to the RNA; wherein the presence of RNA complementary to the oligonucleotide is predictive of the presence or absence of the SNPs described herein.

It should be understood that following any method as described herein related to identifying or screening, such methods may further comprise additional testing or screening for one or more additional genetic mutations, blood tests, blood enzyme tests, cognitive ability tests, counseling, providing support resources or administering an additional pharmaceutical agent based on the results of such tests and/or screens.

It will be understood by those skilled in the art that a wide variety of methods and techniques known in the art may be used in carrying out certain embodiments of the present invention. By way of example, detection of the SNPs described herein may be accomplished using a variety of approaches and techniques well-known in the field, for example those described in U.S. Pat. No. 8,568,968 to Lenz and references cited therein, which are all incorporated by reference in their entirety. Lenz describes various conventional techniques in the art, including those described in:

Sambrook and Russell eds. MOLECULAR CLONING: A LABORATORY MANUAL, 3.sup.rd edition (2001); the series CURRENT PROTOCOLS IN MOLECULAR BIOLOGY (F. M. Ausubel et al. eds. (2007)); the series METHODS IN ENZYMOLOGY (Academic Press, Inc., N.Y.); PCR 1: A PRACTICAL APPROACH (M. MacPherson et al. IRL Press at Oxford University Press (1991)); PCR 2: A PRACTICAL APPROACH (M. J. MacPherson, B. D. Hames and G. R. Taylor eds. (1995)); ANTIBODIES, A LABORATORY MANUAL (Harlow and Lane eds. (1999)); CULTURE OF ANIMAL CELLS: A MANUAL OF BASIC TECHNIQUE (R. I. Freshney 5.sup.th edition (2005)); OLIGONUCLEOTIDE SYNTHESIS (M. J. Gait ed. (1984)); Mullis et al. U.S. Pat. No. 4,683,195; NUCLEIC ACID HYBRIDIZATION (B. D. Hames & S. J. Higgins eds. (1984)); NUCLEIC ACID HYBRIDIZATION (M. L. M. Anderson (1999)); TRANSCRIPTION AND TRANSLATION (B. D. Hames & S. J. Higgins-eds. (1984)); IMMOBILIZED CELLS AND ENZYMES (IRL Press (1986)); B. Perbal, A PRACTICAL GUIDE TO MOLECULAR CLONING (1984); GENE TRANSFER VECTORS FOR MAMMALIAN CELLS (J. H. Miller and M. P. Calos eds. (1987) Cold Spring Harbor Laboratory); GENE TRANSFER AND EXPRESSION IN MAMMALIAN CELLS (S. C. Makrides ed. (2003)) IMMUNOCHEMICAL METHODS IN CELL AND MOLECULAR BIOLOGY (Mayer and Walker, eds., Academic Press, London (1987)); WEIR'S HANDBOOK OF EXPERIMENTAL IMMUNOLOGY (L. A. Herzenberg et al. eds (1996)) all of which are incorporated by reference. Molecular beacons may be used to identify SNPs, as described, for example, by Lenz (U.S. Pat. No. 8,568,968), which further provides, for example, Tyagi and Kramer (1996) Nat. Biotechnol. 14:303-8; Kostrikis (1998) Science 279:1228-9; and Marras (1999) Genet. Anal. 14:151-6; Holland et al. (1991) Proc. Natl. Acad. Sci. 88:7276-7280; and U.S. Pat. No. 5,210,015 by Gelfand et al, all of which are incorporated by reference.

Mass spectrometry techniques may be used to identify SNPs as described herein, for example those described in Mass Spectrometry and Genomic Analysis, ed. Housby, 2001 which is incorporated by reference.

Microarrays may also be used in the detection of SNPs, for example those described in U.S. Pat. No. 8,568,968 to Lenz which include:

Various “gene chips” or “microarray” and similar technologies are known in the art. Examples of such include, but are not limited to LabCard (ACLARA Bio Sciences Inc.); GeneChip (Affymetric, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc.); GEM (Incyte Microarray Systems); a high-throughput microarrying system that can dispense from 12 to 64 spots onto multiple glass slides (Intelligent Bio-Instruments); Molecular Biology Workstation and NanoChip (Nanogen, a microfluidic glass chip (Orchid Biosciences, Inc.); BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu. Rev. Biomed. Eng. 4:129-153. Examples of “gene chips” or a “microarray” are also described in US Patent Publ. Nos.: 2007-0111322, 2007-0099198, 2007-0084997, 2007-0059769 and 2007-0059765 and U.S. Pat. Nos. 7,138,506, 7,070,740, and 6,989,267 all of which are incorporated by reference.

The present invention may employ any of a wide variety of sequencing techniques useful in certain embodiments of the present invention. For example, U.S. Pat. No. 8,568,968 to Lenz provides examples of sequencing techniques and related methods which include: Maxam and Gilbert (1997) Proc. Natl. Acad. Sci. USA 74:560) or Sanger et al. (1977) Proc. Nat. Acad. Sci. 74:5463); Naeve et al. (1995) Biotechniques 19:448; U.S. Pat. No. 5,547,835 and International Patent Application Publication Number WO 94/16101, entitled DNA Sequencing by Mass Spectrometry by Koster; U.S. Pat. No. 5,547,835 and international patent application Publication Number WO 94/21822 entitled “DNA Sequencing by Mass Spectrometry Via Exonuclease Degradation” by Koster; U.S. Pat. No. 5,605,798 and International Patent Application No. PCT/US96/03651 entitled DNA Diagnostics Based on Mass Spectrometry by Koster; Cohen et al. (1996) Adv. Chromat. 36:127-162; and Griffin et al. (1993) Appl. Biochem. Bio. 38:147-159; U.S. Pat. No. 5,580,732 entitled “Method of DNA Sequencing Employing A Mixed DNA-Polymer Chain Probe” and U.S. Pat. No. 5,571,676 entitled “Method For Mismatch-Directed In vitro DNA Sequencing”; U.S. Pat. No. 6,455,249; Cotton et al. (1988) Proc. Natl. Acad. Sci. USA 85:4397; Saleeba et al. (1992) Methods Enzy. 217:286-295 Myers et al. (1985) Science 230:1242); U.S. Pat. No. 6,455,249; Cotton et al. (1988) Proc. Natl. Acad. Sci. USA 85:4397; Saleeba et al. (1992) Methods Enzy. 217:286-295; Orita et al. (1989) Proc. Natl. Acad. Sci. USA 86:2766; Cotton (1993) Mutat. Res. 285:125-144 and Hayashi (1992) Genet. Anal. Tech. Appl. 9:73-79); Keen et al. (1991) Trends Genet. 7:5; Myers et al. (1985) Nature 313:495; Rosenbaum and Reissner (1987) Biophys. Chem. 265:1275; Saiki et al. (1986) Nature 324:163); Saiki et al. (1989) Proc. Natl. Acad. Sci. USA 86:6230 and Wallace et al. (1979) Nucl. Acids Res. 6:3543; Gibbs et al. (1989) Nucleic Acids Res. 17:2437-2448; U.S. Pat. No. 4,998,617 and in Landegren et al. (1988) Science 241:1077-1080. Nickerson et al. (1990) Proc. Natl. Acad. Sci, (U.S.A.) 87:8923-8927; U.S. Pat. No. 5,593,826. in To be et al. (1996) Nucleic Acids Res. 24:3728;U.S. Pat. No. 4,656,127; French Patent 2,650,840; PCT Publication No. WO 91/02087). As in the Mundy method of U.S. Pat. No. 4,656,127; Goelet et al. (PCT Publication No. 92/15712; Cohen et al. (French Patent 2,650,840; PCT Publication No. WO 91/02087; Komher et al. (1989) Nucl. Acids. Res. 17:7779-7784; Sokolov (1990) Nucl. Acids Res. 18:3671; Syvanen et al. (1990) Genomics 8:684-692; Kuppuswamy et al. (1991) Proc. Natl. Acad. Sci. (U.S.A.) 88:1143-1147; Prezant et al. (1992) Hum. Mutat. 1:159-164; Ugozzoli et al. (1992) GATA 9:107-112; Nyren et al. (1993) Anal. Biochem. 208:171-175). Syvanen et al. (1993) Amer. J. Hum. Genet. 52:46-59; U.S. Pat. No. 4,656,127; Cohen et al. (French Patent 2,650,840; PCT Publication No. WO 91/02087; U.S. Pat. No. 4,656,127, all of which are incorporated by reference.

Antibodies may be useful in some embodiments, the production and use of which are well known in the art, for example is described in Current Protocols in Immunology, Coico et al., John Wiley & Sons, which is hereby incorporated by reference.

The references provided herein are all incorporated by reference in their entirety.

The present invention will be further illustrated in the following examples.

### Example 1

Our study focussed on consanguineous families from Pakistan with non-syndromic autosomal recessive intellectual disability (ID).

For a family from the Tehsil Sehnsa District Kotli of Azad Kashmir in Pakistan with 4 affected individuals, we have found a locus in two affected individuals on 11q24.1-q25.

Exome sequencing found homozygous splice donor site (G>A) mutation in intron 4 of DCPS gene (chr11:g.126208295 G>A; NM_{—}014026.3: c.636+1G>A). Other affected individuals were sequenced for same mutation by Sanger sequencing method. We did not observe any homozygous change in these individuals however, one affected individual (V-7) was found to be heterozygous for G>A splice donor change.

Subsequently, by sequencing the other exons of DCPS in individual (V-7), we identified another heterozygous change: NM_{—}014026.3: c.947C>T; p.(Thr316Met).

Affected individuals were diagnosed to have moderate ID, with intelligence quotient (IQ) in the range of 40-50, for all affected individuals.

Other clinical features include mild microcephaly, and muscle weakness in one affected member of the family.

DNAs from 2 affected and 1 unaffected individuals were run on Affymetrix 500K NspI SNP microarray.

11 Mb, 3.5 Mb and 5.2 Mb HBD regions were found on 11q24.1-q25, 14q11.2 and 17q24.2-q24.3, respectively. 14q and 17q loci were excluded by typing polymorphic microsatellite markers in all family members.

Two affected individuals (III-2 and IV-3) has homozygous change (G>A) at splice donor site.

One affected individual (V-7) is compound heterozygous for G>A splice site and Thr316Met.

Both substitutions are not present in >200 Pakistani controls.

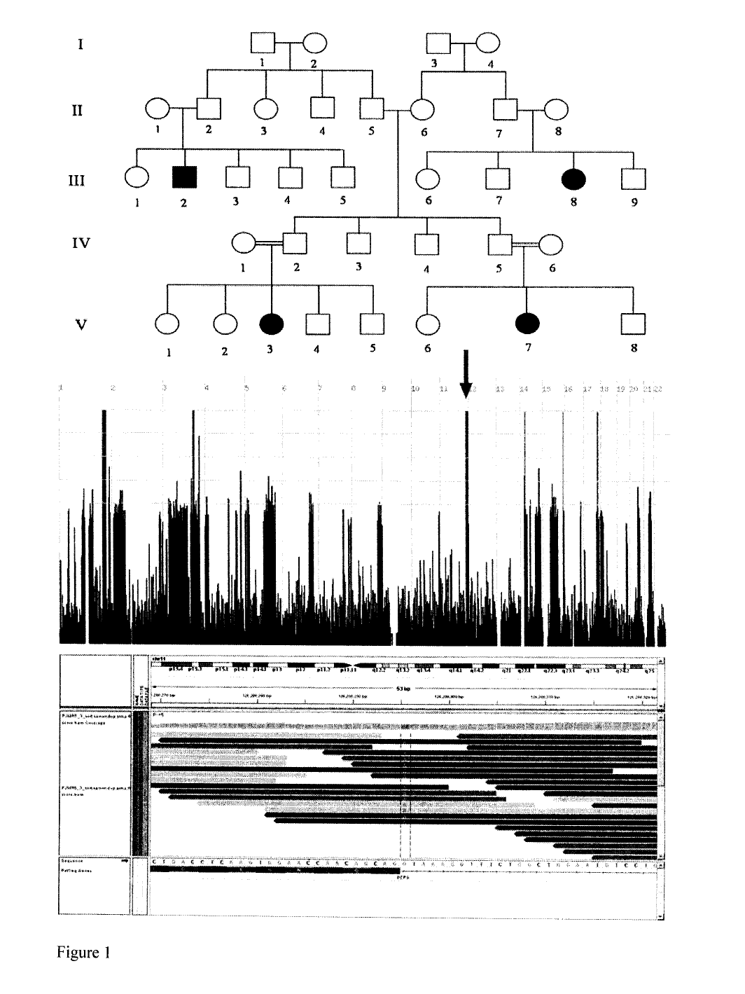

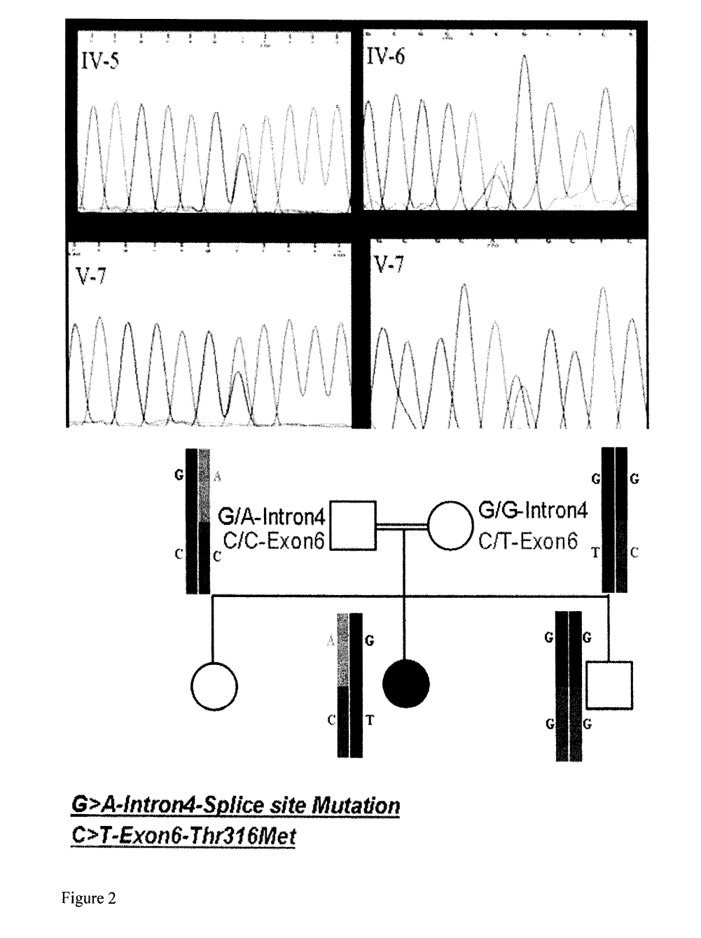

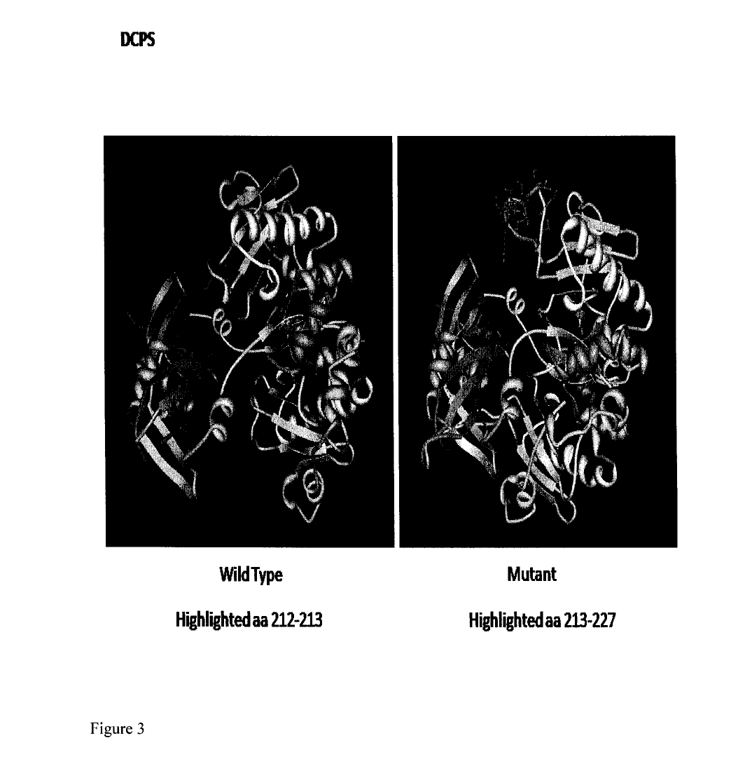

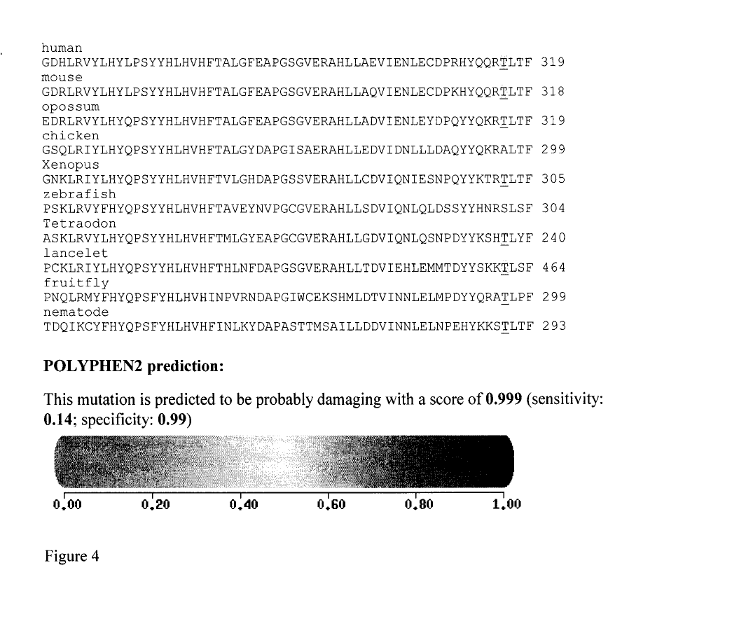

Results of the tests are shown in FIGS. 1-4.

### CONCLUSIONS

Using autozygosity mapping in a consanguineous family from Pakistan, we have identified DCPS on 11q24.2, as a gene for autosomal recessive intellectual disability with additional features including mild microcephaly and mild muscle weakness in one affected individual.

The gene encodes an enzyme known as decapping scavenger that is responsible for mRNA decapping in posttranscriptional processing (including splicing) and release of mature and functional mRNA.

Without wishing to be bound by theory or limiting in any manner, the missense, Thr316Met, and splice site mutations identified are predicted to disrupt crucial protein functions.

### Example 2

### Decapping Activity of Mutant DcpS

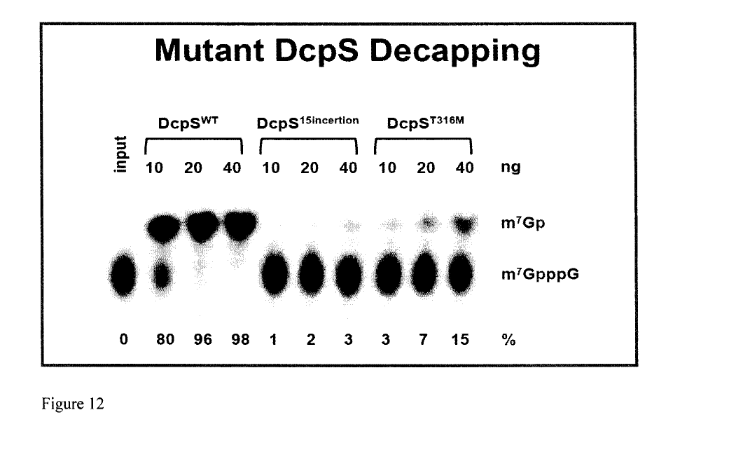

DcpS genes, mutants and normal, were amplified using gene specific primers from TA clones and were sub cloned in expression vector PET-28a and expressed in bacterial BL21(DE3) cells. Enzymatic activity of both mutants and wild type purified DcpS protein was analyzed. DcpS mutant (15 amino acid insertion and Thr316Met) and wild type, at the concentration of 10, 20 and 40 ng, were incubated with 10 fmol of 32-p labelled methylated cap RNA (32-P m7Gp*ppG) substrate (3000 cpm/reaction) at 370 C for five minutes. The reaction products were separated on polyethylenimine (PEI) cellulose thin-layer chromatography (TLC) and developed with 0.45 M (NH4)2SO4. This buffer condition separated the input cap structure from the resulting m7GMP generated by the DcpS decapping activity. Significantly reduced activity was shown by both mutants (the 15 amino acid insertion and Thr316Met) compared to wild type DcpS. The wild type hydrolyzed almost all of 32-P m7Gp*ppG methylated cap structure to methylated guanosine monophosphate (m7Gp) at the lowest concentration (10 ng) within the 5 minutes time point. The mutant with 15 amino acid insertion hydrolyzed only 3% at the highest concentration, whereas, 15% activity was shown by T316M mutant at its highest concentration (FIG. 12).

### Example 3

### Decapping Activity of Mutant DcpS in Whole Cell Extract

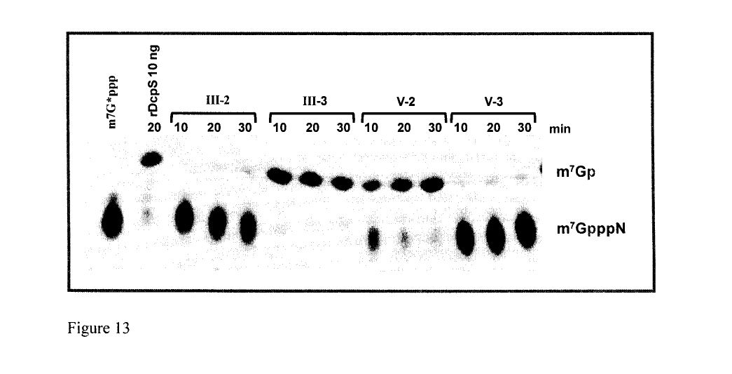

DcpS activity was further investigated in whole cell extract obtained from lymphoblast cell lines of patients and normal individuals. Epstein-Barr virus (EBV) transformed lymphocytes cells now referred as Lymphoblast cell lines were grown in Roswell Park Memorial Institute (RPMI) medium and whole cell extract was prepared for assay. 5 μg of cell extract was incubated at 37° C. with 10 fmol of [32P] cap-labeled RNA (32-P m7Gp*ppG) substrate (3000 cpm/reaction) at the time intervals of 10, 20 and 30 minutes, respectively. Reaction was terminated by incubating reaction mixture tubes in ice. The reaction products were separated on polyethylenimine (PEI) cellulose thin-layer chromatography (TLC) and developed with 0.45 M (NH₄)₂SO₄. DcpS enzyme of patients with 15 amino acid insertion mutation (III-2 and V-3) significantly reduced enzymatic activity compared to heterozygous carrier (II-3 and V-2) and normal control (rDcpS). The mutant DcpS hydrolyzed 6-9% of 32-P m7Gp*ppG methylated cap structure to m7Gp in 30 minutes; whereas, DcpS of heterozygous carrier (III-3 and V-2) and normal displayed 82-87% activity (FIG. 13).

### Example 4

### Expression Levels of DcpS

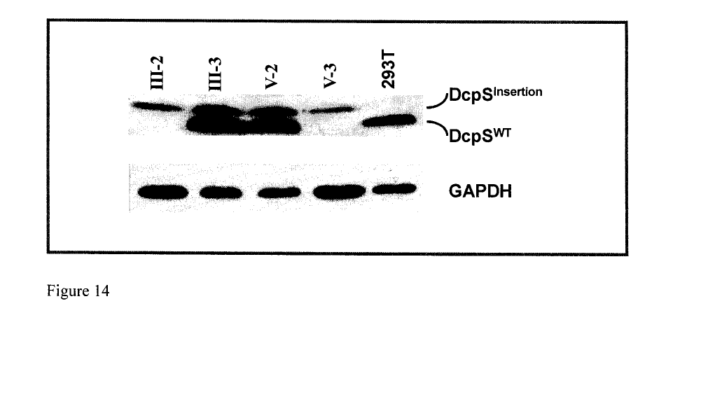

The expression level of DcpS gene was analysed in lymphoblast cell lines of both affected and unaffected individuals through western blotting. The western blot results demonstrated decreased level of expression of DcpS in patients with homozygous 15 amino acid insertions given in lane 01 and 04. The lymphoblast cells carrying heterozygous 15 amino acid insertion mutation is shown in lane 02 and 03; and homozygous normal DcpS in lane 05 (FIG. 14).

Results indicate that homozygous 15aa insert mutation patients have a decreased level of DcpS expression.

Examples 2-4 demonstrate that both mutated DCPS proteins described herein have highly reduced activity. Although these two mutations are exemplified in the examples above, it should be noted that various other DCPS mutations, truncations, insertions, or deletions are also expected to have reduced enzymatic activity in comparison to wild-type.

All citations are hereby incorporated by reference.

The present invention has been described with regard to one or more embodiments. However, it will be apparent to persons skilled in the art that a number of variations and modifications can be made without departing from the scope of the invention as defined in the claims.

1. A Decapping Enzyme Scavenger nucleotide sequence associated with Non-Syndromic Autosomal Recessive Mental Retardation, the nucleotide sequence comprising:
a) at least 7 consecutive nucleotides of SEQ ID NO: 2 and comprising “A” at position 51, or a sequence complementary thereto;
b) at least 7 consecutive nucleotides of SEQ ID NO: 4 and further comprising ATAAAGGTTTCTGGCTGGAATGTCCTGATCTCTGGCCACCCTGCT defined by SEQ ID NO: 13, or a fragment thereof, or a nucleotide sequence that is complementary thereto;
c) at least 7 consecutive nucleotides of SEQ ID NO: 8 and comprising T at position 271, or a sequence complementary thereto, or;
d) at least 7 consecutive nucleotides of SEQ ID NO: 12 and further comprising T at position 947, or a sequence complementary thereto;
e) at least 80% identity to the nucleotide sequence defined in any one of a)-d).

2. The nucleotide sequence of claim 1 wherein the nucleotide sequence comprises at least 15 consecutive nucleotides.

3. The nucleotide sequence of claim 1, wherein the nucleotide sequence comprises at least 7 consecutive nucleotides of SEQ ID NO: 4 and further comprising
ATAAAGGTTTCTGGCTGGAATGTCCTGATCTCTGGCCACCCTGCT defined by SEQ ID NO: 13, or a fragment thereof which is at least 5 continuous nucleotide sequences thereof.

4. The nucleotide sequence of claim 1, which is labeled.

5. A composition comprising the nucleotide sequence of claim 1 further comprising one or more primers that bind to the nucleotide sequence, a thermophilic DNA polymerase, restriction enzyme or any combination thereof.

6. The composition of claim 5, wherein the composition is a nucleotide sequence amplification mixture.

7. A vector comprising the nucleotide sequence of claim 1.

8. A polypeptide defined by SEQ ID NO: 6 or SEQ ID NO: 10.

9. A polypeptide defined as comprising 70% to 100% identity to a) the polypeptide defined by claim 8, b) SEQ ID NO: 6 and comprising a 15 amino acid insertion defined by IKVSGWNVLISGHPA (SEQ ID NO: 14) or c) SEQ ID NO: 10 and comprising Met at position 316.

10. An isolated nucleic acid sequence encoding the polypeptide of claim 9.

11. An antibody that binds to the polypeptide sequence defined by
IKVSGWNVLISGHPA (SEQ ID NO: 14) or SEQ ID NO: 10 when comprising Met at position 3 16.

12. A kit comprising one or more nucleotide sequences defined by claim 1 and optionally any one or combination of a polypeptide, vector, composition or antibody as described herein and optionally further comprising one or more buffers, primers, restriction enzymes, dNTPs, microarrays, gene chips, assay plates, multi-well dishes, glass substrates, purification resins or beads or any combination thereof, wherein the nucleotide sequence, polypeptide, vector, composition or antibody is optionally physically associated or attached to the buffer, primer, restriction enzyme, dNTP, microarray, gene chip, assay plate, multi-well dish, glass substrate, purification resin or bead.

13. A method of detecting or screening a subject for a nucleotide sequence or protein associated with mental retardation comprising,
a) obtaining a biological sample from the subject, the sample comprising genomic DNA, mRNA or protein from the subject;
b) identifying the presence or absence the nucleotide sequence defined by claim 1, wherein the presence of the nucleotide sequence or protein is indicative that the subject has or is at risk for developing cognitive deficits associated with mental retardation or is a carrier for one or more genes associated with mental retardation.

14. The method of claim 13, wherein the step of identifying is performed by microarray analysis, restriction analysis, probe hybridization, nucleotide sequence amplification, PCR, electrophoretic-based nucleic acid analysis, ELISA, DNA sequencing, protein sequencing, antibody binding analysis, mass spectrometry or any combination thereof.

15. A method of detecting or screening a subject for a nucleotide sequence or protein associated with mental retardation comprising,
a) obtaining a biological sample from the subject, the sample comprising genomic DNA, mRNA or protein from the subject;
b) identifying the presence or absence the protein defined by claim 8, wherein the presence of the nucleotide sequence or protein is indicative that the subject has or is at risk for developing cognitive deficits associated with mental retardation or is a carrier for one or more genes associated with mental retardation.

16. The method of claim 15, wherein the step of identifying is performed by microarray analysis, restriction analysis, probe hybridization, nucleotide sequence amplification, PCR, electrophoretic-based nucleic acid analysis, ELISA, DNA sequencing, protein sequencing, antibody binding analysis, mass spectrometry or any combination thereof.
